(19) United States
(12) Patent Application Publication
(10) Pub. No.: US 2014/0066609 A1
(43) Pub. Date: Mar. 6, 2014

(54) SYNTHESIS METHOD OF GLYCO-DRUG RADIOTRACER PRECURSOR

(71) Applicants: Kuei-Lin LU, Taoyuan County (TW); Yu CHANG, Taoyuan County (TW); Cheng-Fang HSU, Taoyuan County (TW); Mei-Hui WANG, Taoyuan County (TW); Wuu-Jyh LIN, Taoyuan County (TW)

(72) Inventors: Kuei-Lin LU, Taoyuan County (TW); Yu CHANG, Taoyuan County (TW); Cheng-Fang HSU, Taoyuan County (TW); Mei-Hui WANG, Taoyuan County (TW); Wuu-Jyh LIN, Taoyuan County (TW)

(73) Assignee: ATOMIC ENERGY COUNCIL-INSTITUTE OF NUCLEAR ENERGY RESEARCH, Taoyuan County (TW)

(21) Appl. No.: 13/604,353

(22) Filed: Sep. 5, 2012

(51) Int. Cl. C07H 15/04

(52) U.S. Cl. 536/17.9

(57) ABSTRACT

A novel synthesis method of Glyco-drug radiotracer precursor is revealed. After completing synthesis of Z-Gly-ah (main structure), galactosamine GalNAc(OAc)₄ is added to have coupling reaction. Then a product is separated directly from dichloromethane. Thus loss of galactosamine during extraction with liquid chromatography is reduced. Moreover, instead of trifluoroacetyl group, carbobenzoxy (abbreviated as Cbz or Z) is used as a protecting group to ensure uniformity of the phase. The cost and synthesis time are also dramatically reduced.

### BACKGROUND OF THE INVENTION

1. Fields of the Invention

The present invention relates to a synthesis method of a Glyco-drug radiotracer precursor, especially to a synthesis method that reduces loss of galactosamine during synthesis processes.

2. Descriptions of Related Art

There are specific receptors on surfaces of human cells and the receptors are used to accept some specific proteins or peptides. According to this specificity, some proteins or peptides are labeled with radioactive nuclides and are delivered into human bodies. Then the labeled proteins or peptides achieve higher concentration in specific organs or tissues so as to diagnose or treat diseases by using nuclear imaging.

There are about two hundred thousand asialoglycoprotein receptors (ASGPR) on surfaces of mammalian heptocytes. The asialoglycoprotein receptor (ASGPR) is a liver-specific transmembrane glycoprotein that mediates endocytosis, removes desialylated glycoproteins, and involves in lipoprotein catabolism. The ASGPR also has high affinity to galactose (Gal) and N-acetylgalactosamine (GalNAc). Especially when a ground substance contains tri-Gals or N-acetylgalactosamine, it has higher affinity to ASGPR on surfaces of hepatocytes, almost 10 times than a substrate with a single saccharide. Based on this characteristic, YEE (ah-GalNAc)₃ has been used as a drug/gene carrier for drug or gene delivery to hepatocytes. Thus characteristic also plays an important role in research and development of radiotracers used in detecting hepatic fibrosis.

The conventional method for synthesis of G-ah-GalNAc is shown in FIG. 1. As shown in figure, D-galactosamine is a staring material to react with pyridine and acetic anhydride to produce a compound GalNAc(OAc)₄ with lower activity. Then GalNAc(OAc)₄ reacts with trimethyl-silyl trifluoromethane sulfonate (TMSOTf) to get an oxazoline derivative with high activity. The reaction time is about 24 hours. The oxazoline derivative is coupled to 6-(trifluoroacetamido)hexanol (TFA-ah) with amino protecting group under catalysis of sulfuric acid to get TFA-ah-GalNAc(OAc)₃. Next perform first liquid chromatography of TFA-ah-GalNAc(OAc)₃ with 90% alcohol for 48 hours.

Under the action of sodium methoxide, the acetyl protecting on the oxygen end is removed. Add alcohol solution containing triethylamine into the solution and stir overnight. Concentrate the solution and purify the solution by using acetic acid aqueous solution. That's the second liquid chromatography. Then vacuum dry the solution. Since acetic acid reacts with hydroxyl group of TFA-ah-GalNAc (OAc)₃ to form esters, add anion exchange resins in hydroxyl form (DOWEX, OH⁻ form) into the solution several times and stir the solution so as to reduce esterification of the hydroxyl group. After removing trifluoroacetyl protecting group on the nitrogen end, ah-GalNAc is obtained.

ah-GalNAc is connected to Glycine with carbobenzoxy (abbreviated as Cbz or Z) protecting group. Then add with DCC and N-Hydroxybenzotriazole (HOBt) in anhydrous N, N-dimethylformamide (DMF) solution into the solution and stir the mixture solution at room temperature overnight. Z-G-ah-GalNAc is obtained by the coupling reaction. At last, the carbobenzoxy protecting group is removed by hydrogenation/reduction to get the final product 6-(glycylamino)hexyl β-N-acetyl-galactosamine (G-ah-GalNAc).

During the synthesis, there are two times of separation by using liquid chromatography. Thus only about 30% amount of GalNAc(OAc)₄ is converted to the final product G-ah-GalNAc. And the total synthesis process takes 30 days. Moreover, the cost other material used in the synthesis such as GalN.HCl (D-galactosamine) is quite expensive. When there is over 50% loss during the liquid chromatography separation process, the optimal control of the cost is getting difficult.

Furthermore, once the hydroxyl protecting group of GalNAc is removed quite earlier, and acetic acid is used as elution solution, the hydroxyl group of GalNAc reacts with acetic acid to have esterification. Thus anion exchange resin in hydroxyl form (DOWEX, OH⁻ form) is used to reduce esterification. Moreover, the hydroxyl-form anion exchange resin has poor efficiency on deacetylation of easters so that stirring overnight is required. This is time-consuming. Furthermore, when the amount of hydroxyl-form anion exchange resins added is too much, pH value of the solution is increased significantly and this leads to the breakage of GalNAc. In addition, HOBt is difficult to be removed by recrystallization. This causes troubles in the following process.

Due to the shortcomings of conventional synthesis method including low efficiency or difficulties in reducing cost, there is room for improvement and a need to provide a novel synthesis method that overcomes the above problems.

### SUMMARY OF THE INVENTION

Therefore it is a primary object of the present invention to provide a synthesis method of Glyco-drug radiotracer precursor that is a simple and integrated method for synthesis of galactopeptide G-ah-GalNAc with lower cost and reduced flow time.

It is another object of the present invention to provide a synthesis method of Glyco-drug radiotracer precursor in which galactosamine GalNAc(OAc)₄ is added and involved in coupling reaction after completing synthesis of the main structure Z-Gly-ah instead of the coupling reaction with Z-Gly after connection of galactosamine to ah. By reducing coupling and involvement of galactosamine, the loss of galactosamine during the synthesis process is reduced.

It is a further object of the present invention to provide a synthesis method of Glyco-drug radiotracer precursor that uses carbobenzoxy (abbreviated as Cbz or Z) as a protecting group during synthesis processes to increase the steric hindrance and ensure uniformity of the phase.

It is a further object of the present invention to provide a synthesis method of Glyco-drug radiotracer precursor that simplifies the synthesis process by removing liquid chromatography purification from the synthesis process. The purification step is deleted due to good solubility of GalNAc and Z-G-ah in dichloromethane. The product with high purity is separated directly from dichloromethane so that the synthesis efficiency is improved.

In order to achieve the above objects, a synthesis method of Glyco-drug radiotracer precursor according to the present invention includes following steps. Firstly synthesize a compound Z-Gly-ah. Synthesize another compound GalNAc(OAc)₄. Then couple Z-Gly-ah to GalNAc(OAc)₄ in a solution containing dichloromethane and a plurality of solvents. Extract the solution. Next dehydrate and concentrate the extracted solution. Add sodium methoxide to the solution for removing an acetyl group of GalNAc(OAc)₄ in the solution. Then adjust pH value of the solution to 6 by using cation exchange resin in the H form. Filter and concentrate the solution. Wash the solution with dichloromethane and filter the solution again to get a compound Z-G-ah-GalNAc. Finally, hydrogenate/reduce the compound Z-G-ah-GalNAc to get a compound G-ah-GalNAc.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 shows steps of a flow chart of a conventional synthesis method;

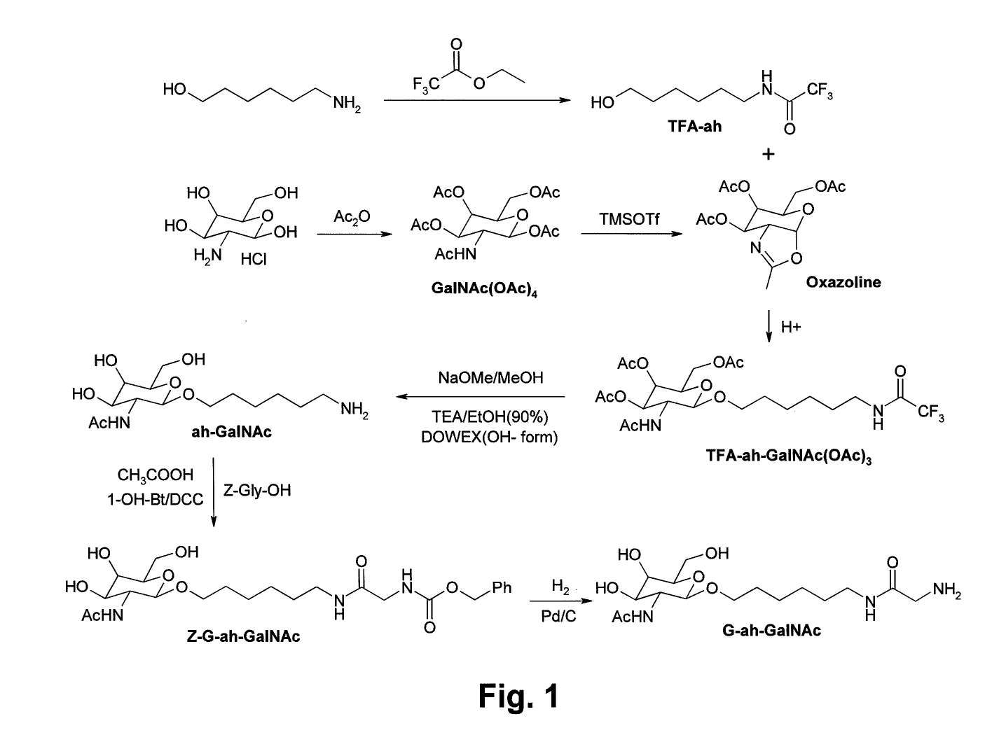

FIG. 2 shows steps of a flow chart of a synthesis method of Glyco-drug radiotracer precursor according to the present invention;

FIG. 3 is a flow chart showing synthesis steps of Z-Gly-ah of an embodiment according to the present invention;

FIG. 4 is a flow chart showing synthesis steps of GalNAc(OAc)₄ of an embodiment according to the present invention;

FIG. 5 is a flow chart showing synthesis steps of Z-G-ah-GalNAc of an embodiment according to the present invention;

FIG. 6 is a flow chart showing synthesis steps of G-ah-GalNAc of an embodiment according to the present invention.

### DETAILED DESCRIPTION OF THE PREFERRED EMBODIMENT

In the conventional synthesis method, the synthesis efficiency is not ideal or the cost is too high due to their shortcomings. Thus the conventional synthesis method is not prevalent. The present invention provides a new synthesis method to overcome these shortcomings.

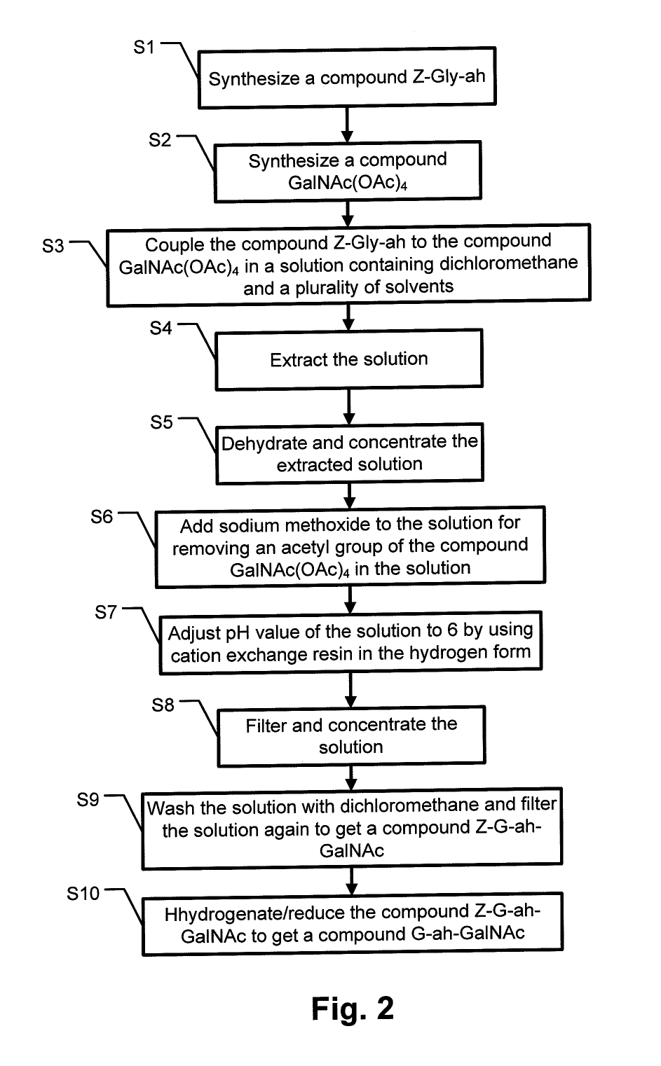

Refer to FIG. 2, it shows steps of a synthesis method of Glyco-drug radiotracer precursor according to the present invention. The synthesis method includes following steps.
Step S1: Synthesize a compound Z-Gly-ah;
Step S2: Synthesize a compound GalNAc(OAc)₄;
Step S3: Couple the compound Z-Gly-ah to the compound GalNAc(OAc)₄ in a solution containing dichloromethane and a plurality of solvents;
Step S4: Extract the solution;
Step S5: Dehydrate and concentrate the extracted solution;
Step S6: Add sodium methoxide to the solution for removing an acetyl group of the compound GalNAc(OAc)₄ in the solution;
Step S7: Adjust pH value of the solution to 6 by using cation exchange resin in the hydrogen form;
Step S8: Filter and concentrate the solution;
Step S9: Wash the solution with dichloromethane and filter the solution again to get a compound Z-G-ah-GalNAc; and
Step S10: Hhydrogenate/reduce the compound Z-G-ah-GalNAc to get a compound G-ah-GalNAc.

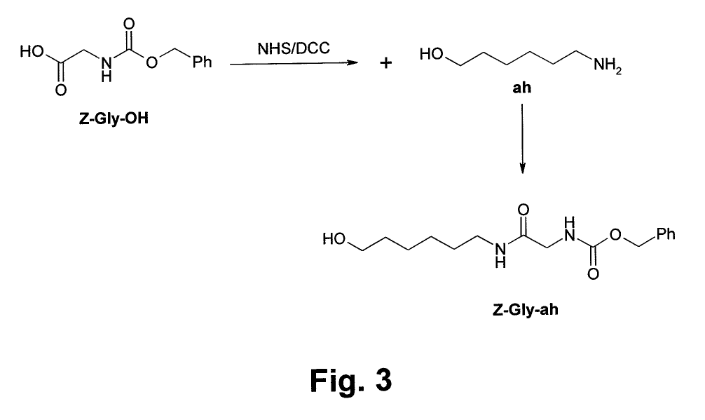

Among these steps, the key point is to simplify and integrate past techniques for synthesis of G-ah-GalNAc. In the step S1, only the compound Z-Gly-ah is synthesized and there is no galactosamine GalNAc(OAc)₄ involved. During synthesis of Z-Gly-ah, refer to FIG. 3, use a compound Z-Gly-OH and N-hydroxysuccinimide (NHS) to react in tetrahydrofuran (THF) solution containing 1, 3-dicyclohexyl-carbodiimide (DCC), and molecular sieve. The solution is stirred at room temperature overnight and then is coupled to 6-aminohexanol (ah) to obtain the compound Z-Gly-ah.

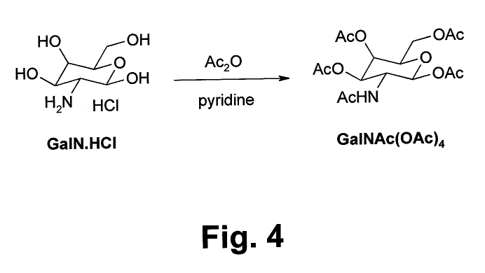

After completing the preparation of Z-Gly-ah, run the step S2 to synthesize the compound GalNAc(OAc)₄. Refer to FIG. 4, use GalN.HCl as a reactant. That means to deal with D-(+)-Galactosamine hydrochloride by using pyridine solution containing acetic anhydride so as to protect hydroxyl groups thereof and get the compound GalNAc(OAc)₄.

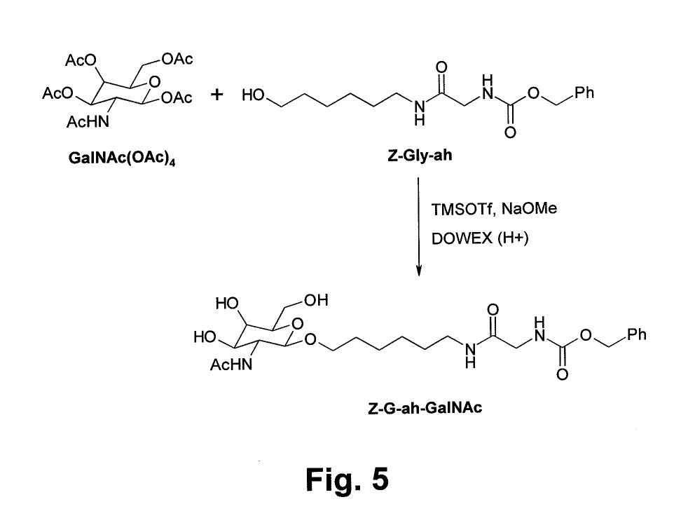

After the synthesis of Z-Gly-ah and GalNAc(OAc)₄, take the step S3 to couple the two compounds. Refer to FIG. 5, Z-Gly-ah and GalNAc(OAc)₄ are reacted in a solution containing a plurality of solvents. In the present invention, a 1, 2-dichloromethane (ethylene dichloride) solution at 50° C. containing TMSOTf, a little amount of DMF, and dichloromethane is used. The solution is stirred overnight to perform the coupling reaction.

From the step S4 to the step S8, the product of the coupling reaction is further processed. After the 1, 2-ethylene dichloride solution being cooled, use triethylamine to neutralize TMSOTf derivative. Then use saturated sodium bicarbonate solution to extract the solution for four times (take the organic layer). Next use sodium chloride aqueous solution to extract once. Also take the organic layer. The organic layer is dehydrated by anhydrous sodium sulfate and then concentrated. The concentrated residue obtained is treated by 0.10M sodium methoxide (NaOMe) to remove the acetyl protecting group. At last, adjust pH value of the solution to 6 by cation exchange resin in hydrogen (H+) form (DOWEX, H⁺ form). That's acidification.

Filter the acidified product again and concentrate. Then the residue is washed by dichloromethane and is filtered again. Collect the solid to get 6-(Benzyloxycarbonylglycylamino) hexyl β-N-acetyl-galactosamine (the compound Z-G-ah-GalNAc) with high purity.

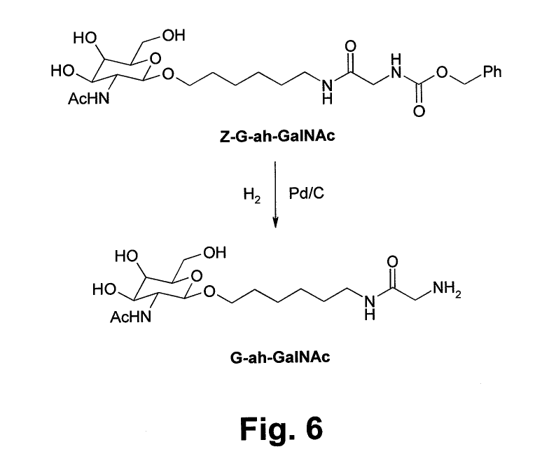

Refer to FIG. 6, in the last step S10, dissolve Z-G-ah-GalNAc in methanol and add a certain amount of 10% palladium carbon (Pd/C) catalyst. Then the solution is vibrated in 50 psi hydrogen gas at room temperature overnight for hydrogenation/reduction. After filtration and concentration, final product G-ah-GalNAc is obtained. The product G-ah-GalNAc doesn't need further separation and purification.

According to the above steps of the present invention, the product the same with the product prepared by conventional techniques is obtained but the yield rate is up to 75%. Moreover, the synthesis cost and time are dramatically reduced. The modification problem of oxazoline with high activity caused by long term synthesis process can also be avoided. The possible loss of galactosamine during the synthesis process is decreased. In the present invention, Z-Gly-ah is synthesized firstly and then galactosamine is directly coupled to Z-Gly-ah. In contrast, galactosamine is connected to ah firstly in conventional synthesis process and then is coupled to Z-Gly. Moreover, Cbz is used as a protecting group to replace TFA. Thus at least 20% phase change caused by TFA protecting group can be prevented and the quality of the product is ensured.

The compound G-ah-GalNAc produced by the present invention is of high yield rate and low cost. Due to high affinity to mammalian heptocytes, G-ah-GalNAc can be applied to preparation of radiotracers, labeling of liver tumors or radiotherapy of liver cancers. The synthesis method of Glyco-drug radiotracer precursor of the present invention has significant medical values.

The followings are details and related parameters of each step according to the present invention.

{synthesis of Z-G-ah, (Benzyl(6-hydroxyhexylcarbamoyl) methylcarbamate)}

Scale 4 g 4A molecular sieve, Cbz-Glycine (Z-Gly-OH, 2.09 g, 10 mmol), N,N′-dicyclohexylcarbodiimide (DCC, 3.09 g, 15 mmole), and N-hydroxysuccinimde (NHS, 1.38 g, 12 mmole) and put them into a 100 ml round-bottom flask and pump to create vacuum for 2 hours. Add 30 ml anhydrous THF into the solution, stir the solution at room temperature overnight. Then suction filter by using a ceramic funnel and take the filtrate, add with 6-aminohexanol (ah, 1.17 g, 10 mmol), and stir the solution at room temperature overnight. Concentrate the solution under reduced pressure and pump to create vacuum overnight. With an ice bath, stir the solution with 300 ml 50% ethyl alcohol for 30 min. Then suction filter by using a ceramic funnel, take the filtrate, add methanol, and concentrate in vacuo and dry at 35□. Set the solution in a vacuum system and pump overnight. Then stir the solution with 150 ml EtOAc for 30 min with an ice bath. Next suction filter by using a ceramic funnel, take the solid. At last, set the solid in a vacuum system and pump overnight to get white solid Z-G-ah (1.4 g, 45%).

### Compound Data of the Product

IR(neat) v_{N—H}=3386 cm⁻¹, 3265 cm⁻¹, v_{C═O}=1690 cm⁻¹, 1650 cm⁻¹.

¹H-NMR(CDCl₃) δ 7.38 (m, 5H, Ph—H), 6.18 (br, 1H, C₆—NH), 5.57 (br, 1H, C₈—NH), 5.12 (s, 2H, C₁₀—H₂), 3.83 (d, 2H, C₈—H₂), 3.63 (t, 2H, C₁—H₂), 3.24 (t, 2H, C₆—H₂), 1.83 (br, 1H, OH), 1.55 (m, 4 H, C₂—H₂ & C₅—H₂), and 1.35 (m, 4 H, C₃—H₂ & C₄—H₂).

¹³C-NMR(CDCl₃) δ 169(C₇), 158(C₉), 137(C₁₀), 128(C₁₂, C₁₃, C₁₄, C₁₅ & C₁₆), 67(C₁₀), 62(C₁), 44(C₈), 39(C₆), 32(C₂), 29(C₅), 27(C₄), and 25(C₃).

MS: m/z 331 [(M+Na)]⁺°

{Synthesis of GalNAc(OAc)₄ (β-N-Acetylgalactosamine per-O-acetate)}

Take D-(+)-Galactosamine hydrochloride [GalN.HCl] (15 g, 69.9 mmol) in a 250 ml round-bottom flask and suspend GalN.HCl in 60 ml anhydrous pyridine and 90 ml, 10.5M acetic anhydride. Stir the solution at room temperature overnight (about 16 hours). Filter the suspension and wash solid with acetic acid and water alternatively. White solid is obtained after being dried. Add 15 ml ether and pump dry to get white solid product GalNAc(OAc)₄(20.4 g, 75%).

### Compound Data Of The Product

IR(neat) v_{N—H}=3230 cm⁻¹, v_{C═O}=1750 cm⁻¹, 1730 cm⁻¹.

¹H-NMR(CDCl₃) δ 5.68 (d, 1H, C₁—H), 5.38 (s, 1H, NH), 5.36 (dd, 1H, C₄—H), 5.06 (dd, 1H, C₃—H), 4.43 (dd, 1H, C₂—H), 4.15 (dd, 1H, C₆—H_{b}), 4.07 (dd, 1H, C₆—Hₐ), 4.00 (td, 1H, C₅—H), 2.15 (s, 3 H, C₈—H₃), 2.11 (s, 3 H, C₁₄—H₃), 2.05 (s, 3H, C₁₂—H₃), 2.03 (s, 3H, C₁₀—H₃) and 2.00 (s, 3 H, C₁₆—H₃).

¹³C-NMR(CDCl₃): δ 170 (C₇, C₉, C₁₁ and C₁₃), 169 (C₁₅), 93 (C₁), 71 (C₅), 70 (C₃), 66 (C₄), 61 (C₆), 49 (C₂), 23 (C₈) and 20 (C₁₀, C₁₂, C₁₄ and C₁₆).

{synthesis of Z-G-ah-GalNAc (6-(Benzyloxycarbonyl-glycylamino)hexyl β-N-acetyl-galactosamine)}

Put GalNAc(OAc)₄ (1.2 g, 3.1 mmol) and Z-G-ah (1.14 g, 3.7 mmol) into a 250 ml round-bottom flask and pump to create vacuum for 2 hours. Add 1, 2-ethylene dichloride (100 ml), dichloromethane (20 ml) and anhydrous N, N-dimethylformamide (DMF, 4 ml) into the solution. Then add trimethylsilyl trifluoromethane sulfonate (TMSOTf, 0.7 ml) to form a brick red solution that is delivered to a drying tube, heated to 50 degrees Celsius and stir the solution overnight. After being cooled down to room temperature, the solution is added with 1 ml triethylamine and is stirred for 5 min. Add 5 g NaHCO3 and 50 ml deionized water into the solution and stir the mixture for 5 min. Then wash with saturated sodium bicarbonate solution (100 ml×4) and ice 1N sodium chloride solution (100 ml) in turn. Next the organic layer is added with dichloromethane, concentrated under reduced pressure, put in a vacuum system and pump overnight. Use 300 ml methanol to wash the residue in batches in a 500 ml round-bottom flask. Then add 0.572 ml, 5.4M NaOMe/MeOH into the solution, cover a glass cap, stir the solution at room temperature for 2 hours. Add certain amount of DOWEX 50W×8 (H⁺ form) for adjusting pH value of the solution to 6 (detected by litmus paper) and stir the solution at room temperature for 30 min. Concentrate the solution under reduced pressure and vacuum dry at 25° C. Put the concentrated solution in a vacuum system and pump overnight. With an ice bath, add 150 ml dichloromethane into the solution and stir the solution for 30 min. Suction filter by using a ceramic funnel and take the solid. Put the solid in a vacuum system and pump overnight to get light brown solid product Z-G-ah-GalNAc (1.18 g, 75%).

### Compound Data of the Product

IR(KBr) v_{N—H}=3377 cm⁻¹, v_{C═O}=1747 cm⁻¹, 1657 cm⁻¹.

¹H-NMR (CD₃OD) δ 7.35 (m, 5 H, Ph—H), 5.08 (s, 2 H, C₁₈—H₂), 4.36 (d, 1H, C₁—H), 3.83 (m, 3H, C₂—H, C₃—H, C₄—H), 3.71 (m, 4H, C₆—H₂ & C₁₆—H₂), 3.55 (dd, 1H, C₅—H), 3.43 (t, 2 H, C₉—H₂), 3.20 (t, 2 H, C₁₄—H₂), 1.94 (s, 3H, C₈—H₃), 1.51 (m, 4H, C₁₀—H₂, and C₁₃—H₂), and 1.34 (m, 4 H, C₁₁—H₂, and C₁₂—H₂).

¹³C-NMR(CD₃OD): δ 173 (C₇), 171(C₁₅), 157 (C₁₇), 137 (C₁₉), 128 (C₂₀, C₂₂, and C₂₄), 127 (C₂₁, and C₂₂), 102 (C₁), 75 (C₅), 72 (C₄), 69 (C₃), 68 (C₉), 67 (C₁₈), 62 (C₆), 51 (C₂), 44 (C₁₆), 39 (C₁₄), 29 (C₁₀) 28 (C₁₃), 26 (C₁₁), 25 (C₁₂), and 23 (C₈).

MS m/z 534 [(M+Na)⁺]°

{synthesis of G-ah-GalNAc (6-(glycylamino)hexyl (3-N-acetyl-galactosamine)}

Dissolve Z-G-ah-GalNAc (6′, 1.18 g, 2.3 mmol) in 50 ml methanol, and add 180 mg 10% Pd/C into solution. Then use a hydrogenation/reduction device to vibrate the solution in 50 psi H₂ overnight. Suction filter the solution by using a ceramic funnel, take the filtrate and concentrate the filtrate under reduced pressure. After vacuum dry overnight, white solid product G-ah-GalNAc (0.96 g, 80%) is obtained.

### Compound Data of the Product

IR(KBr): v_{N—H}=3310 cm⁻¹, v_{C═O}=1650 cm⁻¹.

¹H-NMR (CD₃OD): δ 4.36 (d, 1H, C₁—H), 3.83 (m, 3 H, C₂—H, C3-H, C₄—H), 3.71 (m, 2 H, C₆—H₂), 3.55 (dd, 1H, C₅—H), 3.43 (t, 2 H, C₉—H₂), 3.30 (t, 2H, C₁₆—H₂), 3.20 (t, 2H, C₁₄—H₂), 1.94 (s, 3 H, C₈—H₃), 1.51 (m, 4H, C₁₀—H₂, and C₁₃—H₂), and 1.34 (m, 4 H, and C₁₂—H₂).

¹³C-NMR(CD₃OD): δ 173 (C₇ & C₁₅), 102 (C₁), 75 (C₅), 72 (C₄), 69 (C₃), 68 (C₉), 61 (C₆), 53 (C₂), 43 (C₁₆), 39 (C₁₄), 33 (C₁₀), 29 (C₁₃), 26 (C₁₁), 25 (C₁₂), and 23 (C₈).

MS: m/z 400 [(M+Na)⁺], and 378 [(M+1)⁺]°

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A synthesis method of Glyco-drug radiotracer precursor comprising the steps of:
synthesizing a compound Z-Gly-ah that is represented by the following structural formula: 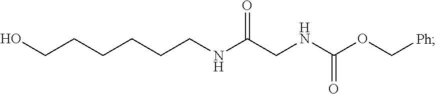 [SMILES: O=C(CNC(=O)OCc1ccccc1)NCCCCCCO]
synthesizing a compound GalNAc(OAc)₄ that is represented by the following structural formula: 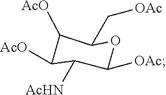 [SMILES: CC(=O)NC1C(OC(C)=O)OC(COC(C)=O)C(OC(C)=O)C1OC(C)=O]
coupling Z-Gly-ah to GalNAc(OAc)₄ in a solution containing dichloromethane and at least one solvent;
extracting the solution;
dehydrating and concentrating the extracted solution;
adding sodium methoxide into the solution for removing an acetyl group of GalNAc(OAc)₄ in the solution;
adjusting pH value of the solution to 6 by using hydrogen-form cation exchange resin, filtering and concentrating the solution;
washing the solution with dichloromethane and filtering the solution again to get a compound Z-G-ah-GalNAc that is represented by the following structural formula: 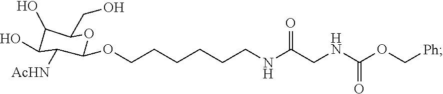 [SMILES: CC(=O)NC1C(OCCCCCCNC(=O)CNC(=O)OCc2ccccc2)OC(CO)C(O)C1O]
and
hydrogenating/reducing the compound Z-G-ah-GalNAc to get a compound G-ah-GalNAc that is represented by the following structural formula:

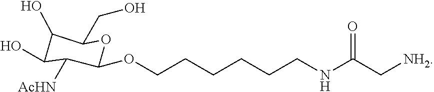 [SMILES: CC(=O)NC1C(OCCCCCCNC(=O)CN)OC(CO)C(O)C1O]

2. The method as claimed in claim 1, wherein the step of synthesizing the compound Z-Gly-ah further includes the step of
treating Z-Gly-OH with 1, 3-dicyclohexylcarbodiimide (DCC) and N-hydroxysuccinimide (NHS) and then to be coupled to 6-aminohexanol (ah) to get the compound Z-Gly-ah;
wherein Z-Gly-OH that is represented by the following structural formula:

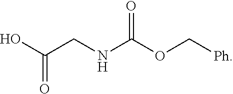 [SMILES: O=C(O)CNC(=O)OCc1ccccc1]

3. The method as claimed in claim 1, wherein the step of synthesizing a compound GalNAc(OAc)₄ further includes a step of:
treating a compound GalN.HCl by using pyridine solution containing acetic anhydride to get the compound GalNAc(OAc)₄;
wherein the compound GalN.HCl is represented by the following structural formula:

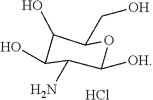 [SMILES: NC1C(O)OC(CO)C(O)C1O ; Cl]

4. The method as claimed in claim 1, wherein the at least one solvent is selected from the group consisting of trimethyl-silyl trifluoromethane sulfonate (TMSOTf), dimethylformamide (DMF) and 1, 2-dichloromethane.

5. The method as claimed in claim 1, wherein the step of extracting the solution further includes the steps of:
using saturated sodium bicarbonate solution to extract the solution for four times; and
using sodium chloride solution to extract once.

6. The method as claimed in claim 1, wherein in the step of dehydrating and concentrating the extracted solution, the dehydrating is performed by using anhydrous sodium sulfate.
